(19) United States
(12) Patent Application Publication
(10) Pub. No.: US 2024/0125759 A1
(43) Pub. Date: Apr. 18, 2024

(54) SYSTEMS AND METHODS FOR REAL-TIME MEASUREMENT OF SOIL CARBON SEQUESTRATION USING NON-INVASIVE MULTIMODAL SENSORS

(71) Applicant: GROUNDTRUTH AG, INC., Arlington, VA (US)

(72) Inventors: John Richard Anderson, Jr., Raleigh, NC (US); Graham Hunter Bowers, Raleigh, NC (US); Lars Dyrud, Great Falls, VA (US); Clayton Raynor Honeycutt, Roseboro, NC (US); Jacob Samuel Lasky, Fort Collins, CO (US); Christopher Casey Nobblitt, Durham, NC (US)

(73) Assignee: GROUNDTRUTH AG, INC., Arlington, VA (US)

(21) Appl. No.: 18/548,194

(22) PCT Filed: Mar. 8, 2022
(86) PCT No.: PCT/US2022/019295

§ 371 Date: Aug. 28, 2023

Related U.S. Application Data
(60) Provisional application No. 63/158,755, filed on Mar. 9, 2021.

(51) Int. Cl. G01N 33/24; G01V 11/00; G06N 20/00

(52) U.S. Cl. CPC G01N 33/24; G01V 11/002; G06N 20/00; G01N 2033/243; G01N 2033/245

(57) ABSTRACT

A system of soil carbon measurement is provided. The system includes a multimodal sensor payload that includes multiple sensors that are each a different sensor type and that are configured to estimate quantities of carbon stored in a soil area. The sensors include multiple stand-off sensor technologies.

### CROSS REFERENCE TO RELATED APPLICATIONS

The present PCT application claims domestic priority to U.S. Provisional Patent Application No. 63/158,755, filed on Mar. 9, 2021, the disclosure and content of which are incorporated by reference herein in their entirety.

### FIELD

The present disclosure relates to soil management and health, and, in particular, to provide soil carbon measurement.

### BACKGROUND

Widespread concern about climate change, greenhouse gas emissions, soil health and food security has prompted many public and private organizations to advocate and adopt carbon sequestration strategies for working lands. In carbon sequestration programs, crop producers and land managers are paid for trapping CO₂ in soil organic matter. They may be compensated for incremental tons of carbon stored in their fields.

Increases in soil carbon can be achieved by transitioning to no-tillage crop production or some variation of no-till farming. In tandem with no-till farming, utilization of cover crops and animal wastes may increase carbon sequestration while improving overall soil health and environmental quality. Atmospheric carbon can also be captured in soil by land managers who convert working lands to grasslands or other uses that give rise to incremental soil carbon storage.

Considerable effort has been expended to measure carbon sequestration via remote sensing from manned and/or unmanned aircraft and satellites. Such complex methods may focus consistently upon measurement of biomass or “carbon stock” and indirect modeling of carbon sequestration. Given that a single tillage event on the farm can discredit those calculations, soil carbon sequestration measurement and monitoring may revert to and/or rely upon traditional soil sampling methods and subsequent laboratory analysis of soil samples for bulk density and carbon.

A metric ton of carbon stockpiled in soil equates to a “carbon credit” that can be traded in carbon markets. Prior to compensation for carbon credits, carbon levels in farm fields may be measured and certified. Historically and conventionally, credits for carbon stored in soil may be validated via a lengthy, expensive process that involves: (a) collection of soil samples for analysis of carbon concentration from different soil segments, e.g. 0 cm-15 cm and 15 cm-30 cm, (b) collection of additional soil samples to determine soil depth, i.e. the root space and volume of soil from which plants draw water and nutrients, (c) shipment of those samples to a distant laboratory, (d) preparation of those samples and determination of soil carbon concentration and soil depth using labor-intensive protocols supported by statistical modeling that estimates tons of stored carbon per field.

The approaches described in the Background section could be pursued, but are not necessarily approaches that have been previously conceived or pursued. Therefore, unless otherwise indicated herein, the approaches described in the Background section are not prior art to the claims in this application and are not admitted to be prior art by inclusion in the Background section.

### SUMMARY

Some embodiments herein are directed to systems, methods and apparatus that perform operations described herein.

Other methods, computer program products, devices and systems according to embodiments of the present disclosure will be or become apparent to one with skill in the art upon review of the following drawings and detailed description. It is intended that all such additional methods, computer program products, and systems be included within this description, be within the scope of the present disclosure, and be protected by the accompanying claims. Moreover, it is intended that all embodiments disclosed herein can be implemented separately or combined in any way and/or combination.

### BRIEF DRAWING DESCRIPTION

FIG. 1 is a schematic rendering of a system for real-time measurement of soil carbon sequestration using multimodal stand-off sensors according to some embodiments.

FIG. 2 is a schematic block diagram illustrating a system for real-time measurement of soil carbon sequestration using non-invasive multimodal sensors according to some embodiments.

FIG. 3 is a schematic block diagram illustrating a system as described in FIG. 2 including an airborne vehicle according to some embodiments.

FIG. 4 is a schematic block diagram illustrating an electronic configuration for a computer according to some embodiments.

FIG. 5 is a flowchart of operations for training and using a machine learning model for operations according to some embodiments disclosed herein.

FIG. 6 is a schematic rendering of a system for real-time measurement of soil carbon sequestration using multimodal stand-off sensors according to some embodiments.

### DETAILED DESCRIPTION

In the following detailed description, numerous specific details are set forth in order to provide a thorough understanding of embodiments of the present disclosure. However, it will be understood by those skilled in the art that the present invention may be practiced without these specific details. In other instances, well-known methods, procedures, components, and circuits have not been described in detail so as not to obscure the present invention. It is intended that all embodiments disclosed herein can be implemented separately or combined in any way and/or combination.

Some embodiments of the present invention include scalable methods that employ non-invasive, stand-off technologies to detect, visualize and quantify intra- and inter-field agricultural soil carbon sequestration in near real time. Embodiments may provide economic and environmental advantages to farmers and other land owners because they provide an initial approach in a novel solution to the expanding problem of determining soil carbon sequestration. Embodiments herein may enable land owners to confidently determine soil carbon sequestration in real-time using non-invasive techniques.

Widespread concern about climate change, greenhouse gas emissions, soil health and food security has prompted many public and private organizations to advocate and adopt carbon sequestration strategies for working lands. In carbon sequestration programs, crop producers and land managers are paid for trapping CO₂ in soil organic matter. They are compensated for incremental tons of carbon stored in their fields.

An example of the basic equation used to calculate metric tons of carbon stored in a finite area of soil is shown below:

10,000 m² in one hectare×0.1 m soil depth×1.4 g/cm³ bulk density×1.2% Carbon=16.8 t/ha.

It is important to note that the quantity of carbon stored in soil, as calculated in the above equation, may require three values, one each for soil carbon concentration, soil bulk density and soil depth. In practice, values for those equation parameters may be interpolated and/or derived from analysis of soil samples. For example, one method is to take one sample per 5 acres for soil carbon concentration analysis. Sampling resolution for soil bulk density and soil depth determination may typically be less. In some instances, those samples that are more difficult to collect and/or those that are more expensive to collect may be taken at a reduced spatial density such as, for example, every 10-20 acres.

As a general matter, soil carbon concentration, soil depth and soil bulk density values may vary significantly in and/or across fields. Thus, meaningful errors in the estimation of those parameters can be generated if soil sampling is performed poorly and/or if an insufficient number of soil samples is collected. Additionally, the equation used to calculate sequestered carbon may be overly simplistic because it may fail to adequately and robustly describe soil depth and volume, a potentially large source of error in the computation of how much carbon is stored in soil.

Given: (a) the time, expense and physical difficulty of collecting soil samples and (b) the relatively small number of soil samples used to quantify soil carbon concentration, soil bulk density and soil depth in carbon sequestration programs, frequent errors may occur in the calculation of carbon sequestered in soils. In carbon markets, such errors may have profound financial implications.

Given current measurement methodology and prices paid for sequestered carbon, it is impractical and prohibitively expensive to accurately sample carbon concentration, soil bulk density and soil depth in the hundreds or thousands of fields that may be enrolled in future carbon sequestration programs. Suffice it to say that scaling carbon measurement methods for expanding carbon sequestration programs is a daunting challenge.

Ground-penetrating radar (GPR) signals can be strongly correlated with soil bulk density and GPR can accurately determine soil depth and volume. Electromagnetic induction, via its detection of electrical conductivity, may offer further insight into soil depth and the volume of soil that may host biological activity.

Recent United States Department of Agriculture (USDA) research studies indicate that inelastic neutron scattering (INS), another air-launched sensor technology, can affordably measure soil carbon and other elemental concentrations with accuracy and precision. In different soil types, carbon determinations with INS correlate well with dry combustion carbon measurements that are the industry standard. Integrated with a global positioning system (GPS) and a geographic information system (GIS), INS can be used to map carbon concentration variations in fields. However, INS alone, may not provide the soil bulk density and depth values needed to validate soil carbon in tons per acre.

An additional stand-off sensor, laser-induced breakdown spectroscopy (LIBS), can be used to measure soil carbon concentrations. Soil carbon estimates obtained with LIBS are also highly-correlated with carbon estimates obtained via dry combustion methods.

Given the rapidly growing demand for carbon sequestration in farm fields, concomitant demand for carbon credit validation and the compelling need for farmers to understand how their cultural practices affect concentration and spatial distribution of subterranean carbon, carbon industry experts are calling for new carbon sequestration measurement and/or monitoring methods.

In accordance with embodiments herein, highly-mobile, multimodal, stand-off sensors, operating in an edge computing environment equipped with machine learning capabilities may be deployed in farm fields to measure and visualize carbon sequestration. Embodiments may dramatically increase the accuracy and speed at which carbon sequestration in working lands can be measured and monitored. Embodiments may provide 3-dimensional visualization of the soil volume and may illustrate spatial distribution of carbon and soil density fluctuations within the soil volume. Embodiments may advance calculation of soil depth and volume such that new inferences about soil management for carbon sequestration purposes can be drawn. Embodiments may further provide methods and systems that validate carbon credits in (near) real time without collection of expensive soil samples, distant laboratory involvement and long waits for laboratory analysis results. Cost reductions for soil carbon measurement and/or monitoring may be expected.

In some embodiments, a field may be defined as multiple geospatial coordinates that form the boundary for a measurable, three-dimensional area of land. The area of land may contain an uneven mass of soil that supports growth of vegetation and/or that exhibits variable depth and/or density attributes. Some embodiments provide automated methods and/or systems for estimating soil carbon sequestered in a field. Some embodiments include stand-off methods that do not include the traditional and widespread practice of collecting soil samples and sending those samples to a distant laboratory for purposes of estimating soil bulk density and carbon concentration in a field.

Some embodiments include a multimodal payload of integrated, air-launched sensors that may include at least one or more of the following: a ground-penetrating radar sensor (GPR), an electromagnetic induction sensor (EMI), an air-launched inelastic neutron scanning sensor (INS), a laser-induced breakdown spectrometer (LIBS) sensor and a global positioning system (GPS). In some embodiments, the one or more sensors are coupled to either the front or rear of an all-terrain vehicle (ATV). In some embodiments, the one or more sensors may be coupled to other land-traversing vehicles, manned or unmanned farm machinery, robots and/or drones, among others. Some embodiments include an onboard computer that receives, processes and geolocates data received from the one or more sensors. Some embodiments provide that some operations herein may be performed in an edge computing environment in which some data is stored in and/or some calculations are performed by the onboard computer while other data is stored in and/or some calculations are performed in a computing cloud. In some embodiments, the sensors may be hard-wired and/or wirelessly-connected to the onboard computer. Data housed in the onboard computer may be communicated wirelessly to the computing cloud and/or uploaded to the cloud via a portable drive.

In use and operation, as the multimodal sensor payload traverses a field via an ATV and/or other vehicle, the multimodal sensors scan the subterranean soil environment. Some embodiments provide that GPR, EMI and INS return signals are received via the onboard computer and/or the connected computing cloud and are processed using a machine learning model and fused such that depth and density of soil in a field is calculated from the processed GPR, EMI and INS signals.

Some embodiments provide that, in a typical field, soil depth and density may not be constant and thus may vary significantly across landscapes. Accordingly, in some embodiments, the ATV or vehicle hosting the multimodal sensor payload may traverse an entire field numerous times, collecting thousands of data points that account for the field's natural variability in soil depth and density. In this manner, embodiments herein may provide significantly greater sampling resolution for soil density and soil depth than conventional approaches. Some embodiments provide machine learning components that may include forms of artificial intelligence (AI), which may enhance the capability to describe and quantify soil depth and volume and thus calculate sequestered carbon with increased accuracy and precision. In such machine learning models, soil carbon calculation may improve with each acre of data ingested. In this manner, embodiments herein may increase the capability to quantify soil depth, volume and carbon concentration in heterogenous soils.

Inclusion of GPR, EMI and GPS in the multimodal sensor payload, in tandem with machine learning and advanced modeling techniques, may provide an interactive, inch-by-inch, 3-dimensional visualization of the soil volume that depicts uneven spatial boundaries and inherent variations in soil density and carbon concentration. The system and method herein model and mathematically describe the innate soil mass and volume as it is, avoiding the monolithic, simplistic and frequently inaccurate mathematical description of soil and sequestered carbon that arises from traditional soil sampling and use of aforementioned equation.

In some embodiments, a portable LIBS may be substituted for INS in the multimodal sensor payload.

Some embodiments provide systems including a manually- or autonomously-operated all-terrain vehicle (ATV) that is equipped with a location device, such as a global positioning system (GPS). The vehicle may include a hardened, weather-resistant, laptop computer that directs and receives data from a hardware payload comprising two or more sensors that may be complementary, automated, and/or multimodal. The sensors are selected and integrated specifically for the purpose of optimizing geospatial detection and quantification of soil carbon sequestration. The multimodal sensor payload may provide non-invasive hardware designed and operated specifically for the purpose of characterizing soil carbon sequestration.

In some embodiments, the dual or tri-sensor payload may include an airborne (above ground), not coupled-to the-ground, ground penetrating radar (GPR) antenna. The GPR antenna may include a horn antenna, a multi-frequency antenna, an array antenna, a phased array antenna and receiver, among others. Embodiments may further include a source of acoustic waves and a corresponding acoustic wave receiver. The acoustic components of embodiments may be deployed at the soil surface inside agricultural double disk openers, inside a small gauge wheel and/or inside a “knife” normally used to apply liquid fertilizers. In such embodiments, tubes intended to deliver fertilizer to the soil surface may be replaced with data transmission cables connecting the system laptop to acoustic components of the payload.

In some embodiments, as the payload-bearing ATV moves across the soil surface, the radar antenna and receiver may be deployed approximately one foot above the soil surface and the acoustic or seismic components may be deployed at the soil surface. Some embodiments provide that the radar antenna and receiver may be deployed less than one foot or greater than one foot above the soil surface. Data from the radar and acoustic sensors are collected and transmitted directly to the hardened laptop. Some embodiments provide that the data are, via automated algorithms and analytics unique to such embodiments, transformed, fused and combined with GPS coordinates and elevation data such that the location and depth of soil carbon sequestration may be defined. In some embodiments, field by field, the soil carbon sequestration may be determined across a crop production enterprise. Pursuant to these calculations performed by the onboard laptop computer, embodiments may further include a telemetry device connected to the laptop computer. In some embodiments, the telemetry device may transmit the transformed and fused data directly to a multiaccess “edge” cloud computing environment where the data may be deposited into a data lake structure. Some embodiments provide that, in the cloud computing environment, additional algorithms, analytics and machine learning protocols may access and utilize data from the data lake structure to create a visual image of soil carbon sequestration that may be three-dimensional.

The visual image may depict the portions of a geographical area, i.e., a field or landscape in terms of the soil carbon sequestration in that geographical area. Fusion of elevation data, i.e., a digital elevation model, into this visualization of soil carbon sequestration is also performed to provide additional richness to the data. For a farmer, land manager or agronomist, inclusion of elevation data in the data fusion-visualization process puts subsurface soil carbon sequestration into context relative to slope and/or soil type, among others.

Use of the onboard laptop to perform the calculative workload and immediate movement of that mathematical work product into the aforementioned multi-access cloud computing environment via the onboard telemetry device gives the present embodiments extremely low latency. Additional calculations may be performed and data transformation may occur in the cloud computing environment. In this manner, a farmer or interested party can, via an internet interface and mobile telephone, tablet and/or computer, view soil carbon sequestration within a field, among fields in a farming unit, across a landscape or throughout an entire crop production enterprise. Given the computational design and telemetry integrated into the present embodiments, soil carbon sequestration may be characterized in real to near real time.

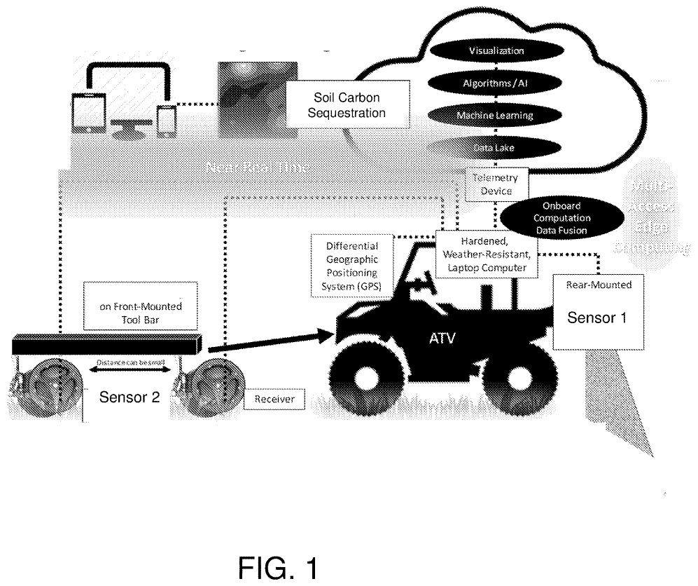

Reference is now made to FIG. 1, which is a schematic rendering of a system for real-time measurement of soil carbon sequestration using multimodal stand-off sensors according to some embodiments. As illustrated, a vehicle, such as an all-terrain vehicle (ATV) may have a first sensor (Sensor 1) that is mounted to the rear thereof. Sensor 1 may include any number of sensor technologies including but not limited to GPR, EMI, INS and/or LIBS, among others. In some embodiments, a second sensor (Sensor 2) or third sensor (Sensor 3) may be mounted to the front or rear of the vehicle. These sensors may include any number of sensor technologies, including but not limited to GPR, EMI, INS and/or LIBS, among others. Some embodiments provide the Sensor 1 and Sensor 2 and Sensor 3 include sensor technologies that are different from one another.

In some embodiments, sensor 1 and/or sensor 2 may comprise an integrated a multimodal stand-off sensor unit that includes, for example, GPR, EMI, INS and/or LIBS sensors.

In some embodiments, a computing device may be supported by the vehicle and may receive and/or store sensor data that is received from Sensor 1 and/or Sensor 2 and/or Sensor 3. Some embodiments provide that the computer comprises a hardened weather-resistant laptop computer, but such embodiments are non-limiting as the computer may include a different form factor including mobile telephone, tablet, software defined radio and/or fixedly mounted computer.

A location and/or navigation device may be provided in the vehicle and may generate geographic location information corresponding to the vehicle and/or sensor data. For example, some embodiments provide that the location and/or navigation device comprises a differential geographic positioning system (GPS). Location data from the location and/or navigation device may be provided to the computer. In some embodiments, the computer may associate the location data with the sensor data that is received from Sensor 1 and/or Sensor 2 and/or Sensor 3. In this manner, the data corresponding to each location that is traversed by the vehicle may be determined to provide location specific soil carbon sequestration data.

A telemetry device may transmit the location specific soil carbon sequestration data from the computer to a remote computer and/or data repository using any combination of wired and/or wireless communication protocols and/or technologies. In some embodiments, the remote computer may perform additional analysis and may generate a three-dimensional soil carbon sequestration map corresponding to the location specific soil carbon data among others.

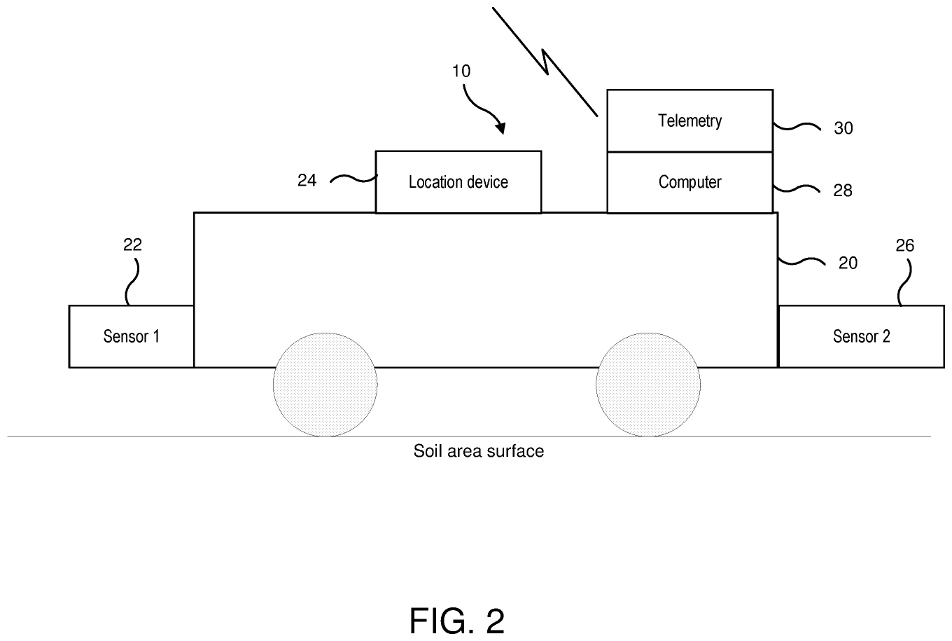

Reference is now made to FIG. 2, which is a schematic block diagram illustrating a system for real-time measurement of soil carbon sequestration using non-invasive multimodal sensors according to some embodiments. A system 10 according to some embodiments includes a vehicle 20 that is configured to travel over a soil area. A location device 24 is configured to provide geographic location data corresponding to the vehicle 20. At least one sensor 22, 26 is mounted to the vehicle to cause the at least one sensor to move above a surface of the soil area as the vehicle travels thereon and to generate data relating to a physical, chemical and/or biological characteristic of the soil corresponding to the soil area. Depending on the sensor technology, the at least one sensor 22 (Sensor 1) may include a non ground-coupled sensor. A computer 28 is communicatively coupled to the at least one sensor 22, 26 and to the location device 24. The computer may be configured to receive the geographic location data and the data relating to the physical, chemical and/or biological characteristic of the soil. The computer 28 may be further configured to generate location associated data relating to the physical, chemical and/or biological characteristic of the soil corresponding to the soil area. In some embodiments, the data relating to the physical, chemical and/or biological characteristic of the soil correlates to soil depth, soil volume, soil density and to carbon concentration. Such data may be combined to generate soil carbon sequestration at specific geolocated points in the field and may be aggregated to provide a soil carbon sequestration value corresponding to a given area.

Some embodiments provide that the soil area includes multiple soil area elements. For example, a soil element may correspond to a specific area, size and/or shape of the soil surface. In some embodiments, each soil area element corresponds to a specific geographic location and a corresponding location associated with a soil carbon sequestration data value. Some embodiments provide that each soil area element includes an area that is in a range from about one square foot to about ten acres. Such embodiments are non-limiting examples, however, as a soil element may be less than one square foot and/or larger than ten acres.

In some embodiments, a sensor 22, 26 comprises a ground penetrating radar (GPR). Some embodiments provide that the GPR is configured to operate in a frequency range of about 10 MHz to about 5 GHz. Such embodiments are non-limiting examples, however, as the operational frequency range may be less than 10 MHz or greater than 5 GHz. In some embodiments, the GPR is configured to operate in a frequency range of about 100 MHz to about 600 MHz. In some embodiments, the GPR is configured to operate in a frequency range of about 200 MHz to about 800 MHz. In some embodiments, the GPR is configured to operate at or above about 100 MHz. In some embodiments, the GPR is configured to operate at or below about 800 MHz. Some embodiments provide that the GPR is configured to operate in VHF, UHF and/or L-Band frequency ranges. In some embodiments, the GPR is a non ground-coupled antenna. Some embodiments provide that the non ground-coupled antenna includes a horn antenna and/or an array antenna.

In some embodiments, the GPR is configured to operate in a plurality of different frequency ranges. Some embodiments provide that the GPR is configured to simultaneously operate in different frequency ranges.

In some embodiments, at least one sensor 22, 26 is a non-invasive sensor relative to the surface of the soil area. Some embodiments provide that at least one sensor 22, 26 is configured to move in a range from at the surface of the soil area to about six feet above the surface of the soil area. However, such range is non-limiting as the sensor 22, 26 may be configured to operate at an elevation that is higher than six feet relative to the soil surface.

Some embodiments include a sensor support that is configured to physically support at least one sensor 22, 26 and to be pulled and/or pushed across the surface of the soil area by the vehicle 20. In some embodiments, the sensor support is and/or includes a self-propelled vehicle that is separate from the vehicle or towed vehicle that is coupled to the vehicle. Some embodiments provide that the location associated soil carbon sequestration data includes elevation data corresponding to the soil carbon sequestration data.

In some embodiments, the at least one sensor 22, 26 includes EMI, INS and/or LIBS sensors which may be used in combination with the GPR and GPS sensors.

In some embodiments, the vehicle comprises a self-driving vehicle and is configured to traverse the soil area in a path that is defined by a coverage plan that is based on the geographic location data. For example, a terrestrially operating vehicle such as a self-driving ATV, cart, or tractor may use the location data in conjunction with a coverage plan to traverse the soil are in the predefined path.

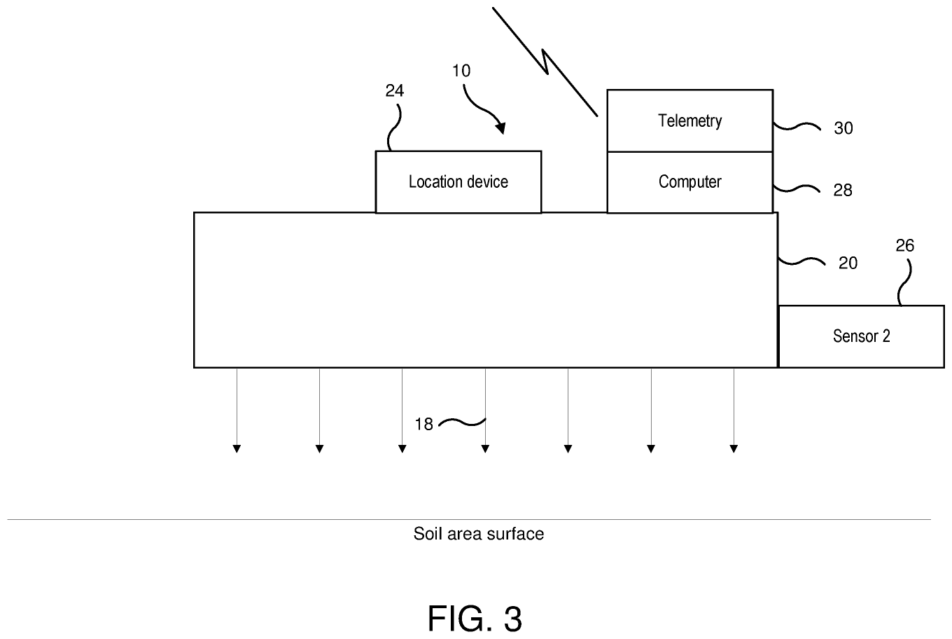

Brief reference is now made to FIG. 3, which is a schematic block diagram illustrating a system as described in FIG. 2 including an airborne vehicle according to some embodiments. In some embodiments, the vehicle comprises an airborne vehicle and is configured to fly over the soil area based on self-generated lift 18. In some embodiments, the airborne vehicle is an autonomously flying drone that operates according to a predefined coverage plan that may define elevation, speed and path. Some embodiments provide that the drone is tethered to a ground station and/or another vehicle while other embodiments provide that the drone is untethered. In some embodiments, the drone may include telemetry 30 for transmitting the generated data during and/or after flight. Some embodiments provide that the drone include on board memory for storing the generated data.

In some embodiments, the airborne vehicle is configured to fly over the soil area in a pattern that is defined by a coverage plan that is based on the geographic location data. Although the airborne vehicle is illustrated as including only a second sensor 26, embodiments herein provide that such sensor is a multimodal sensor including one or more of GPR, EMI, INS and/or LIBS sensors which may be used in combination with the GPS sensor.

Referring back to FIG. 2, some embodiments provide that the computer 28 is further configured to generate a single soil carbon sequestration value for the soil area. that is based on the location associated soil carbon sequestration data. In some embodiments, at least one sensor 22, 26 is a non-invasive sensor and the soil carbon sequestration data is graphically represented based on data generated using the non-invasive multimodal sensor.

Some embodiments provide that the computer 28 is located at the vehicle and that a second computer is remote from the vehicle 20. The computer 28 may be further configured to generate the location associated soil carbon sequestration data and to transmit the location associated soil carbon sequestration data to a data repository that is accessible by the second computer. In some embodiments, the second computer is configured to receive the location associated soil carbon sequestration data.

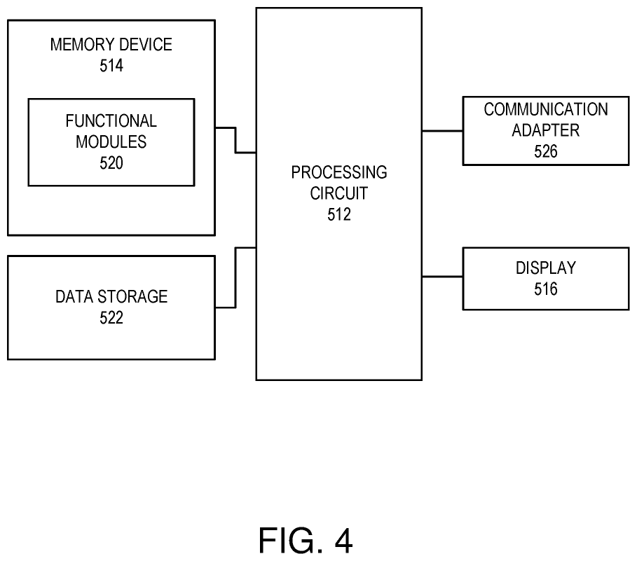

Reference is now made to FIG. 4, which is a schematic block diagram illustrating an electronic configuration for a computer according to some embodiments. As shown in FIG. 4, the computer 28 may include a processing circuit 512 that controls operations of the computer 28. Although illustrated as a single processing circuit, multiple special purpose and/or general-purpose processors and/or processor cores may be provided in the computer 28. For example, the computer 28 may include one or more of a video processor, a signal processor, a sound processor and/or a communication controller that performs one or more control functions within the computer 28. The processing circuit 512 may be variously referred to as a “controller,” “microcontroller,” “microprocessor” or simply a “computer.” The processor may further include one or more application-specific integrated circuits (ASICs).

Various components of the computer 28 are illustrated as being connected to the processing circuit 512. It will be appreciated that the components may be connected to the processing circuit 512 through a system bus, a communication bus and controller, such as a USB controller and USB bus, a network interface, or any other suitable type of connection.

The computer 28 further includes a memory device 514 that stores one or more functional modules 520.

The memory device 514 may store program code and instructions, executable by the processing circuit 512, to control the computer 28. The memory device 514 may also store other data such as image data, event data, user input data, and/or algorithms, among others. The memory device 514 may include random access memory (RAM), which can include non-volatile RAM (NVRAM), magnetic RAM (ARAM), ferroelectric RAM (FeRAM) and other forms as commonly understood in the gaming industry. In some embodiments, the memory device 514 may include read only memory (ROM). In some embodiments, the memory device 514 may include flash memory and/or EEPROM (electrically erasable programmable read only memory). Any other suitable magnetic, optical and/or semiconductor memory may operate in conjunction with the gaming device disclosed herein.

The computer 28 may further include a data storage device 522, such as a hard disk drive or flash memory. The data storage device 522 may store program data, player data, audit trail data or any other type of data. The data storage device 522 may include a detachable or removable memory device, including, but not limited to, a suitable cartridge, disk, CD ROM, DVD or USB memory device.

Embodiments herein may include neural networks for processing and/or analyzing data. Some embodiments herein may rely on one or more algorithms including statistical and/or machine learning techniques. Such labelling techniques may include, but are not limited to, labeling of data with semi-supervised classification, labeling of data with unsupervised classification, DBSCAN, and/or K-means clustering, among others. Such classification techniques may include, but are not limited to linear models, ordinary least squares regression (OLSR), stepwise regression, multivariate adaptive regression splines (MARS), locally estimated scatterplot smoothing (LOESS), ridge regression, least absolute shrinkage and selection operator (LASSO), elastic net, least-angle regression (LARS), logistic regression, decision tree, other tree-based algorithms (e.g. ADA-Boost), support vector machine, and neural network based learning. Neural network-based learning may include feed forward neural networks, convolutional neural nets, recurrent neural nets, long/short term memory neural, auto encoders, generative adversarial networks [especially for synthetic data creation], radial basis function network, and any of these can be referred to as “deep” neural networks. Additionally, ensembling techniques to combine multiple models, bootstrap aggregating (bagging), random forest, gradient boosted models, and/or stacknet may be used.

Additionally, in some embodiments, training data may optionally be transformed using dimension reducing techniques, such as principal components analysis, among others.

Laser-induced breakdown spectroscopy. To accelerate collection and measurement of soil nutrient levels, some embodiments use LIBS, a stand-off, laser-based technology that has, to date, been used, for the most part, to detect metallic elements in civil engineering and industrial applications. Some embodiments include portable LIBS units. Laser-induced breakdown spectroscopy has been adapted for use in aqueous environments and, in the laboratory, it has been used to measure elements in soil. Some embodiments provide that LIBS can measure elements that are essential to a crop plant as well as elements customarily found on a soil test report. In addition, LIBS has been used to estimate soil carbon, a viable surrogate for OM values found on soil test reports. In some embodiments, LIBS may be used to measure soil nutrients, in situ, in a farm field. Some embodiments provide that automated LIBS are used, in either multimodal or autonomous fashion, for agricultural purposes.

Ground-Penetrating Radar. Ground-penetrating radar may be used to detect the presence of soil roots for plant phenotyping purposes.

Electromagnetic Induction. Electrical conductivity measurements obtained with an EMI sensor may be used to designate management zones within fields, i.e. intra-field areas that are contiguous and similar enough in texture and water holding capacity to be managed as a single entity.

Some embodiments provide a mobile, self-propelled, soil health and management laboratory (MSHML). It can be operated autonomously or manually. A multimodal trifecta of sensors may be deployed in combination. The MSHML payload comprises simultaneous use of ground-penetrating radar (GPR), laser-induced breakdown spectroscopy (LIBS) and electromagnetic induction (EMI) sensors, deployed, in this case, to collect and fuse information about physical, chemical and biological characteristics of soil. Embodiments provide a data upload capability and communications link that connects the MSHML to a cloud computing environment.

In some embodiments, placement of these particular sensors, GPR, INS, EMI and LIBS, onto an autonomous, all-terrain vehicle (ATV), and integration of those sensors with other digital technologies, on and off the ATV constitute an automated, stand-off method for assessing soil health and quality. Via the machine and methods presented herein, one can collect, transmit and display reliable information about physical, chemical and biological characteristics of soil in near real time, in effect, delivering essential information a farmer needs to manage for a healthy soil. Some embodiments provide a near real time assessment of soil health, delivered in a context suitable for crop producer use. In some embodiments, the MSHML is a self-propelled suite of devices, sensors and technologies used in combination for the purpose of monitoring soil health. The machine consists of an ATV that can be operated manually or autonomously. The ATV may transport an automated, multimodal payload consisting of GPR, LIBS, INS and EMI sensors. Other components on the ATV are integrated with the stacked sensor payload. Components include a power source, an electrical converter, a computer hardened for outdoor use, a differential global positioning system (GPS), a conventional or multispectral camera and a wireless data communication system. Collectively, the “stacked” sensor payload and these elements provide near real time wireless transmission of data describing physical, chemical and biological characteristics of soil into a cloud computing enterprise.

Some embodiments use commercial technology to wirelessly transmit data directly into a computing environment architecture, such as a hybrid enterprise cloud, the enterprise being a data lake, i.e., a database configuration that manages structured and unstructured data, supports visual analytics and facilitates machine learning focused upon below ground attributes of soil. Therein, computer code, algorithms and analytics fuse data from the respective sensors to generate unique visualizations and assessments relevant to soil health and management.

In some embodiments, in a directed sampling mode, responding to wireless commands from its laptop control station, the machine moves to the desired latitude and longitude in a farm field. In some embodiments, the MSHML uses a nearest neighbor, statistical algorithm that considers historical productivity, elevation and other parameters to select optimum sampling sites. Finally, the MSHML can be programmed to grid sample, i.e., to collect measurements at coordinates corresponding to a grid, e.g., the 2.5-acre to 5.0-acre grid that is commonly used for variable rate fertilizer application.

In some embodiments, the GPR sensor is mounted beneath the ATV and connected to the onboard computer that receives its instructions from a laptop control station. Upon reaching the proper coordinates in either directed or grid sampling mode, the automated MSHML collects soil carbon sequestration data using its GPR sensor, operating at, for example, 500 MHZ.

In some embodiments, a processing device, such as the computer 28 referenced in FIGS. 2-4, may be removable and/or fixably mounted to and/or supported by a vehicle 20. In some embodiments, the processing circuit 512 may be configured to receive, from a location device, geographic location data corresponding to a location of the vehicle. The processing circuit 512 may be further configured to receive, from a sensor that is proximate the vehicle, data relating to a physical, chemical and/or biological characteristic of a soil area. The processing circuit 512 may further generate location associated data that relates the geographic location data to the physical, chemical and/or biological characteristic of the soil area at respective locations corresponding to the geographic location data.

Some embodiments provide that the sensor is caused to move above a surface of the soil area as the vehicle travels thereon and to generate the physical, chemical and/or biological characteristic data corresponding to the soil area. In some embodiments, the data relating to the physical, chemical and/or biological characteristic of the soil includes electrical conductivity.

In some embodiments, the soil area includes multiple soil area elements that may each correspond to a specific geographic location and a corresponding location associated soil carbon sequestration data value. Some embodiments provide that each soil area element includes an area that is in a range from about one square foot to about ten acres.

In some embodiments, the first and/or second sensor 22, 26 includes a ground penetrating radar (GPR) that may be configured to operate in a frequency range of about 10 MHz to about 5 GHz. In some embodiments, the GPR is configured to operate in a frequency range of about 200 MHz to about 800 MHz. In some embodiments, the GPR is configured to operate at or above about 100 MHz. In some embodiments, the GPR is configured to operate at or below about 800 MHz. Some embodiments provide that the GPR is configured to operate in VHF, UHF and/or L-Band frequency ranges. Some embodiments provide that the GPR is a non ground-coupled antenna that may include a horn antenna and/or an array antenna.

In some embodiments, the first and/or second sensor 22, 26 is a non-invasive sensor relative to the surface of the soil area. For example, some embodiments provide that the first and/or second sensor 22, 26 are configured to provide data without directly contacting the corresponding soil area. Some embodiments provide the first and/or second sensor 22, 26 is configured to move in a range from at the surface of the soil area to about six feet above the surface of the soil area.

Some embodiments further include a sensor support that is configured to physically support the first and/or second sensor 22, 26 and to be propelled across the surface of the soil area by the vehicle 20.

In some embodiments, the location associated data includes location associated soil carbon sequestration data that includes elevation data corresponding to soil carbon sequestration.

In some embodiments, the vehicle 20 is a self-driving vehicle and is configured to traverse the soil area in a path that is defined by a coverage plan that is based on the geographic location data. Some embodiments provide that the vehicle 20 is an airborne vehicle and is configured to fly over the soil area based on self-generated lift. In some embodiments, the airborne vehicle is configured to fly over the soil area in a pattern that is defined by a coverage plan that is based on the geographic location data.

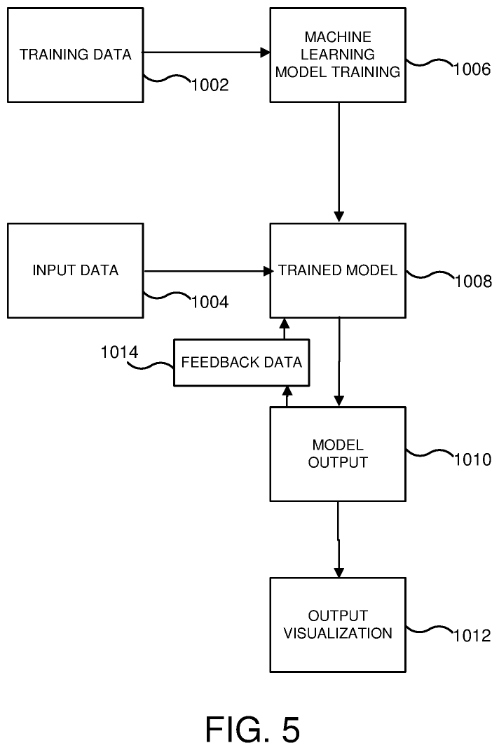

Reference is now made to FIG. 5 which is a flowchart of operations for training and using a machine learning model for operations according to some embodiments disclosed herein. Some embodiments provide that training data (block 1002) is provided to a machine learning platform as disclosed herein. The machine learning platform may perform machine learning model training using the training data that is provided (block 1006). The training data may include data from GPS, GPR, INS, EMI and/or LIBS, among others. The training data values may all be georeferenced according to some embodiments herein. In some embodiments, training data may include air and/or ground temperature, volumetric moisture content, digital elevation model images, soil probe results, penetrometer readings, core samples, acoustic in-situ measurements, in-situ ultrasound measurements, and/or excavation analysis, among others. The machine learning model may be trained using any of the techniques described herein, including, for example, random forest, among others. The result of the training may include a trained machine learning model (block 1008).

Once the machine learning model is trained, input data 1004 may be provided to the model, which may generate model output data 1010. The input data 1004 may include GPR, INS, LIBS and EMI scans and the trained model 1008 may determine soil carbon sequestration data at a high density of locations within a given area.

The model output data 1010 may be used to generate an output visualization (block 1012). For example, the values that are above/below a soil carbon sequestration threshold may be marked in a first color while the values that are not above/below the soil carbon sequestration threshold may be marked with a second color that is different from the first color in the visualization.

In some embodiments, the model output data 1010 may be used as feedback 1014 that may be provided to the trained model 1008 to increase the performance of the trained model 1008.

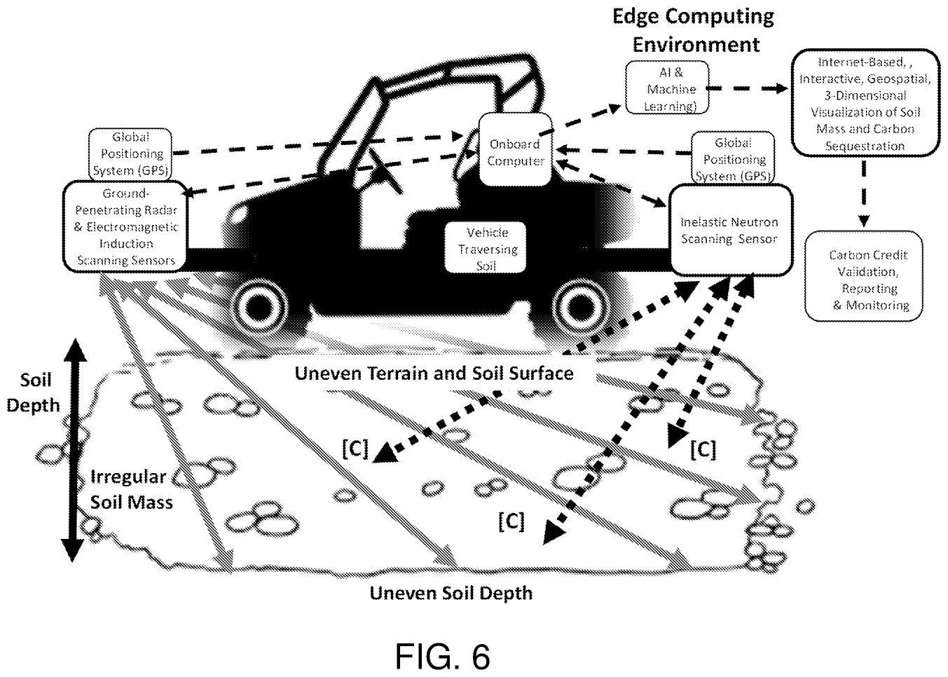

Reference is now made to FIG. 6, which is a schematic rendering of a system for real-time measurement of soil carbon sequestration using multimodal stand-off sensors according to some embodiments. As illustrated, a GPR may scan and accurately measure soil depth. In some embodiments, the GPR may generate a spatially-accurate estimation of soil volume. Some embodiments provide that the GPR scans and generates a spatially accurate estimate of soil bulk density.

In some embodiments, the EMI further scans and generates data that may be included in the three-dimensional soil volume and soil bulk density calculations. In some embodiments, the data from the EMI is fused with the GPR data in a machine learning model to generate the three-dimensional soil volume and soil bulk density calculations.

Some embodiments provide that the INS scans and accurately measures the carbon concentration in the soil. The GPS may geo-locate data from the GPR, EMI and INS. The carbon concentration from the INS is spatially located in the soil mass. In some embodiments, data from the GPS, GPR, INS and/or EMI may be received from an on-board computer. In some embodiments, the received data may be analyzed using artificial intelligence and/or machine learning techniques using the on-board computer and/or in an edge computing environment.

In this manner, the static soil depth and static soil bulk density values (from soil samples) in the carbon sequestration equation may be replaced with spatially-accurate, sensor-acquired values that take into account whole field variability of soil depth and density. In some embodiments, the soil carbon sequestration data may be provided via an internet-based, interactive, geospatial, and/or three-dimensional visualization, among others.

### Further Definitions and Embodiments

In the above description of various embodiments of the present disclosure, aspects of the present disclosure may be illustrated and described herein in any of a number of patentable classes or contexts including any new and useful process, machine, manufacture, or composition of matter, or any new and useful improvement thereof. Accordingly, aspects of the present disclosure may be implemented in entirely hardware, entirely software (including firmware, resident software, micro-code, etc.) or combining software and hardware implementation that may all generally be referred to herein as a “circuit,” “module,” “component,” or “system.” Furthermore, aspects of the present disclosure may take the form of a computer program product comprising one or more computer readable media having computer readable program code embodied thereon.

Any combination of one or more computer readable media may be used. The computer readable media may be a computer readable signal medium or a computer readable storage medium. A computer readable storage medium may be, for example, but not limited to, an electronic, magnetic, optical, electromagnetic, or semiconductor system, apparatus, or device, or any suitable combination of the foregoing. More specific examples (a non-exhaustive list) of the computer readable storage medium would include the following: a portable computer diskette, a hard disk, a random access memory (RAM), a read-only memory (ROM), an erasable programmable read-only memory (EPROM or Flash memory), an appropriate optical fiber with a repeater, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device. Program code embodied on a computer readable signal medium may be transmitted using any appropriate medium, including but not limited to wireless, wireline, optical fiber cable, RF, etc., or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C#, VB.NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable instruction execution apparatus, create a mechanism for implementing the functions/acts specified in the flowchart and/or block diagram block or blocks.

These computer program instructions may also be stored in a computer readable medium that when executed can direct a computer, other programmable data processing apparatus, or other devices to function in a particular manner, such that the instructions when stored in the computer readable medium produce an article of manufacture including instructions which when executed, cause a computer to implement the function/act specified in the flowchart and/or block diagram block or blocks. The computer program instructions may also be loaded onto a computer, other programmable instruction execution apparatus, or other devices to cause a series of operational steps to be performed on the computer, other programmable apparatuses or other devices to produce a computer implemented process such that the instructions which execute on the computer or other programmable apparatus provide processes for implementing the functions/acts specified in the flowchart and/or block diagram block or blocks.

It is to be understood that the terminology used herein is for the purpose of describing particular embodiments only and is not intended to be limiting of the invention. Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of this specification and the relevant art and will not be interpreted in an idealized or overly formal sense unless expressly so defined herein.

The flowchart and block diagrams in the figures illustrate the architecture, functionality, and operation of possible implementations of systems, methods, and computer program products according to various aspects of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular aspects only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Like reference numbers signify like elements throughout the description of the figures.

The corresponding structures, materials, acts, and equivalents of any means or step plus function elements in the claims below are intended to include any disclosed structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The aspects of the disclosure herein were chosen and described in order to best explain the principles of the disclosure and the practical application, and to enable others of ordinary skill in the art to understand the disclosure with various modifications as are suited to the particular use contemplated.

1-42. (canceled)

43. A system of soil carbon measurement comprising:
a multimodal sensor that includes a plurality of sensors that are each a different sensor type and that are configured to estimate quantities of carbon stored in a soil area, wherein the plurality of sensors comprises a plurality of stand-off sensor technologies configured to generate sensor data without contacting the soil area, the multimodal sensor comprising a ground penetrating radar (GPR) sensor and an inelastic neutron scattering (INS) sensor; and
a mobile platform that is configured to support the multimodal sensor.

44. The system of claim 43, wherein the multimodal sensor further comprises an electromagnetic induction (EMI) sensor configured to determine carbon concentration of soil at different soil depths beneath a geographical area defined by a plurality of geospatial coordinates.

45. The system of claim 43, wherein the multimodal sensor further comprises a laser-induced breakdown spectrometer (LIB S) sensor configured to determine carbon concentration of soil at different depths inside a geographical area defined by a plurality of geospatial coordinates.

46. The system of claim 43, wherein the multimodal sensor further comprises a global positioning sensor (GPS) that is configured to geo-locate data of other sensor signals of the multimodal sensor.

47. The system of claim 43, wherein the mobile platform comprises an all-terrain vehicle (ATV), a passenger vehicle, a truck, a farm machine, a robot and/or a drone.

48. The system of claim 43, further comprising an on board computer that is communicatively coupled to the multimodal sensor, wherein the on-board computer is wired to the multimodal sensor and/or is wirelessly coupled to the multimodal sensor.

49. The system of claim 48, wherein the on-board computer is communicatively coupled to a cloud computing environment, wherein the cloud computing environment comprises an edge computing environment that includes an artificial intelligence processing circuit that includes machine learning components.

50. The system of claim 49, wherein the machine learning components are configured to train a GPR sensor to identify soil bulk density.

51. The system of claim 49, wherein the machine learning components are configured to train a GPR sensor to identify soil volume and portions of that volume where crop root growth and biological activity are likely to occur.

52. The system of claim 49, wherein the machine learning components are configured to train an EMI sensor to identify soil volume and portions of that volume where crop root growth and biological activity are likely to occur.

53. A system comprising:
a vehicle that is configured to travel over a soil area;
a location device that is configured to provide geographic location data corresponding to the vehicle;
at least one sensor that is caused to move above a surface of the soil area as the vehicle travels thereon and to generate data relating to a physical, chemical and/or biological characteristic of the soil corresponding to the soil area, wherein the soil area comprises a plurality of soil area elements, wherein each soil area element corresponds to a specific geographic location and a corresponding location associated soil carbon sequestration data value; and
a computer that is communicatively coupled to the at least one sensor and to the location device, that is configured to receive the geographic location data and the data relating to the physical, chemical and/or biological characteristic of the soil, and to generate location associated soil carbon sequestration data corresponding to the soil area.

54. The system of claim 53, wherein the at least one sensor comprises a ground penetrating radar (GPR).

55. The system of claim 54, wherein the GPR comprises a non ground-coupled antenna.

56. The system of claim 54, wherein the at least one sensor comprises at least one of EMI, INS and LIBS

57. The system of claim 53, wherein the computer comprises a first computer that is located on the vehicle and a second computer that is remote from the vehicle,
wherein the first computer is further configured to generate the location associated soil carbon sequestration data and to transmit the location associated soil carbon sequestration data to a data repository that is accessible by the second computer, and
wherein the second computer is configured to receive the location associated soil carbon sequestration data.

58. A method comprising:
using a multimodal sensor and machine learning to generate a 3-dimensional model of the soil volume in a geographical area defined by a plurality of spatial coordinates.

59. The method of claim 58, further comprising determining, using the 3-dimensional model, a variable lower boundary and/or a variable upper boundary of the soil volume that is capable of storing carbon and/or supporting biological activity.

60. The method of claim 59, further comprising generating a spatial distribution of soil density in the soil volume.

61. The method of claim 59, further comprising calculating the soil volume beneath a geographical area defined by a plurality of geospatial coordinates.

62. The method of claim 59, further comprising fusing spatial characteristics of the soil volume with the spatial distribution of soil density to derive soil mass beneath a geographical area defined by a plurality of geospatial coordinates.
